(19) United States
(12) Patent Application Publication
(10) Pub. No.: US 2016/0089470 A1
(43) Pub. Date: Mar. 31, 2016

(54) Purifier

(71) Applicant: SUZHOU BEIANG TECHNOLOGY LTD., Jiangsu (CN)

(72) Inventors: Hongyu Ran, Suzhou (CN); Yan Zhang, Suzhou (CN); Zhaofeng Wei, Suzhou (CN); Yigang Liu, Suzhou (CN)

(21) Appl. No.: 14/890,806

(22) PCT Filed: May 12, 2014
(86) PCT No.: PCT/CN2014/077269

§ 371 Date: Nov. 12, 2015

(30) Foreign Application Priority Data
May 13, 2013 (CN) 201320257584.1
Jun. 4, 2013 (CN) 201310219023.7
Jun. 4, 2013 (CN) 201320318690.6

(51) Int. Cl. A61L 9/22; H01J 27/02

(52) U.S. Cl. CPC A61L 9/22; H01J 27/022

(57) ABSTRACT

Described is an ion air purifier. The ion air purifier includes a collector module (1) and a repeller module (2) which are connected in an openable and closable manner, wherein several collector plates (11) are uniformly distributed on the collector module (1), and several repeller plates (21) are uniformly distributed on the repeller module (2); and each collector plate (11) and each repeller plate (21) are alternately arranged when the two plates are in a closed state. Thus, an optimized distance between adjacent collector plates (11) and repeller plates (21) is ensured; and when the collector module (1) and the repeller module (2) are in an opened state, the collector plates (11) are separated from the repeller plates (21), and the distance between two adjacent collector plates (11) and the distance between two adjacent repeller plates (21) are both increased, thereby facilitating cleaning of the collector module. Further described is a network air purifier.

### CROSS-REFERENCE TO RELATED APPLICATIONS

The patent application claims the priorities of Chinese patent application No. 201320257584.1 submitted on May 13, 2013, Chinese patent application No. 201310219023.7 submitted on Jun. 4, 2013, and Chinese patent application No. 201320318690.6 submitted on Jun. 4, 2013, and the full texts of these applications are incorporated in the present application by reference.

### FIELD OF THE INVENTION

In some embodiments, the present invention relates to the field of indoor purification devices, particularly to an ion air purifier, and in other embodiments, the present invention relates to the technical field of the Internet of things, particularly to a network air purifier.

### BACKGROUND

Increasingly higher life quality has been required in recent years with the increase of economic income and development of the society. While much attention has been paid to scientific and healthy diets, comfortable living environments, and convenient traffic conditions etc., requirements for air quality have become one of the most important factors in people's life quality. Especially, frequent hazes in recent years and a proposed PM2.5 air quality standard have further raised concerns over air quality in life.

It is extremely urgent to develop a high performance air purifier to purify indoor air and optimize indoor environments, and ion air purifiers have been applied more and more widely in everyday life.

A typical ion air purifier comprises an air inlet, an air outlet, and a generator and a collector arranged between the air inlet and the air outlet. The generator and the collector are arranged oppositely. Air reaches the generator via a primary filter screen of the air inlet and the generator forms a plasma field under the action of a high pressure and ionizes some air molecules. When passing through the plasma field, harmful substances comprising bacteria etc. are oxidized and killed by high energy free radicals while macromolecule organic matters comprising formaldehyde etc. are decomposed into water and carbon dioxide by the high energy free radicals. The partly ionized air is accelerated under the action of a force of an electric field to collide with other particles so that the particles are charged, and most dust and bacteria in a box of the purifier are charged through such a series of snowballing avalanche effect. Charged dust grains move in the collector under the action of the force of the electric field and are finally absorbed by the collector with opposite charges. After charges are neutralized, clean air keeps its kinetic energy to continue to advance, thereby forming ion air to circulate air of a whole space.

It may be learned from the working process above that the collector is one of the core components of the ion air purifier, and dust grains in the purifier are absorbed on the surface of the collector. Therefore, it is necessary to purify the collector regularly so as to keep the surface clean and ensure a relatively high adsorption capacity of the collector. However, since the distance between the collector and two adjacent repelling plates thereof needs to satisfy a requirement of optimized distribution of the force of the electric field, the distance therebetween is relatively short, and the collector is difficult to clean.

Therefore, it has become a problem to be solved by those skilled in the art to improve the structure of the collector while ensuring a relatively short distance between adsorption plates of the collector without affecting the adsorption effect so that the collector is convenient to clean.

Besides, various indoor air purifiers have been launched one after another by many manufacturers in order to meet requirements on indoor air quality. However, when using an existing air purifier, a user needs to walk to device to control the air purifier manually with a button or a touch screen etc., thus remote control and information interaction of the air purifier can be hardly implemented and inconvenience is brought when the air purified is used. For example, a user will expect to enjoy fresh air as soon as he/she arrives home instead of turning on an air purifier at home after he/she arrives home.

### SUMMARY

A technical problem to be solved by the present invention is to provide an ion air purifier, which ensures an optimized distance of a force of an electric field between adsorption plates while facilitating cleaning.

The present invention provides an ion air purifier in order to solve the technical problem above, comprising a collector module and a repeller module, which are connected in an openable and closable manner; several collector plates parallel to each other are uniformly distributed on the collector module, and several repeller plates parallel to each other are uniformly distributed on the repeller module; the collector module and the repeller module are isolated electrically, and each collector plate and each repeller plate are alternately arranged when the two plates are in a closed state.

Preferably, a rotating shaft is provided on one end of the collector module, and the repeller module is opened relative to the collector module around the rotating shaft.

Preferably, the collector module and the repeller module may be separated and split completely by opening a fixing member.

Preferably, one end of the collector module away from the rotating shaft is provided with a fixing member, and the collector module and the repeller module are fixedly connected via the fixing member.

Preferably, the fixing member is a hasp.

Preferably, the fixing member is detachably installed on the collector module and the repeller module.

Preferably, when the collector module and the repeller module are in a closed state, adjacent collector plates and repeller plates are mutually insulated with a potential difference, and the ratio of the potential difference to the distance between the adjacent collector plates and repeller plates is smaller than 10⁷ V/m.

The present invention provides an ion air purifier. The ion air purifier comprises a collector module and a repeller module, which are connected in an openable and closable manner, wherein several collector plates are uniformly distributed on the collector module, and several repeller plates are uniformly distributed on the repeller module; and each collector plate and each repeller plate are alternately arranged when the two plates are in a closed state. When the collector module and the repeller module are in a closed state, the collector plates are combined with the repeller plates, that is, detachable collector plates and repeller plates are included. The distance between adjacent collector plates and repeller plates is a set optimized distance. When the collector module and the repeller module are in an opened state, the collector plates are separated from the repeller plates, and the distance between two adjacent collector plates and the distance between two adjacent repeller plates are both increased. Such a detachable design not only ensures an optimized distance between adsorption plates, but also facilitating cleaning of the collector module.

In a preferred embodiment, the ion air purifier provided by the present invention comprises a rotating shaft. The rotating shaft is provided on one end of the collector module, and the repeller module may be opened relative to the collector module around the rotating shaft under the action of an external force, thereby opening the two plates to facilitate cleaning, while preventing the two plates from being separated to facilitate locating when the plates are installed again.

In another specific embodiment, a fixing member is provided on one end of the collector module provided by the present invention away from the rotating shaft. The collector module and the repeller module are fixedly connected via the fixing member. In this way, the two plates are fixed by the fixing member when the collector module and the repeller module are in the opened state, thus preventing the two plates from being opened irregularly and improving the reliability in installing the two plates.

Another technical problem to be solved by the present invention is to provide a network air purifier so as to implement remote start and configuration of a user and bring convenience to the user.

A network air purifier is characterized in that the network air purifier comprises a network communication module, a controller and a purification processing module;

the network communication module is configured to receive an operation instruction via a network, and send the received operation instruction to the controller;

the controller is configured to send a control signal to the purification processing module according to the operation instruction sent by the network communication module and control the purification processing module to perform a purification operation;

the purification processing module is configured to perform air purification processing according to the control signal sent by the controller.

Preferably, the purifier further comprises a remote control terminal;

the remote control terminal comprises an input sub-module and a network communication sub-module;

the input sub-module is configured to receive an operation instruction inputted by a user, and send the operation instruction to the network communication sub-module;

the network communication sub-module is configured to connect the purifier through a network, and send the operation instruction to the network communication module of the purifier.

Preferably, the remote control terminal further comprises a display sub-module;

the display sub-module is configured to display purification state information sent by the network communication module of the purifier to the network communication sub-module;

the purification state information is information for recording a device working state, which is generated when the purification processing module of the purifier performs the purification processing, or hardware alarm information of the purifier.

Preferably, the purifier further comprises a parameter configuration module;

the parameter configuration module is configured to provide an interactive interface of the purifier to set device parameters of the purifier;

the device parameters of the purifier are a network configuration parameter and a purification working parameter of the purifier;

the purification working parameter is a parameter which is required when the purification processing module of the purifier operates.

Preferably, the purifier further comprises an information sending module;

the information sending module is configured to send the purification state information to the remote control terminal through the network communication module; the remote control terminal is provided with a display module to display the received purification state information;

the purification state information is sent to a maintenance server via the network communication module, and recorded by the maintenance server.

Preferably, the purifier further comprises a system maintenance module;

the system maintenance module is configured to perform a maintenance operation to the purifier according to system maintenance information received by the network communication module;

the system maintenance information comprises the purification working parameter of the purifier.

Preferably, the purifier further comprises a storage module;

the storage module is configured to store the purification state information sent by the purification processing module.

Preferably, the network communication module is a wireless network card. Preferably, the purifier further comprises a filter module;

the filter module is configured to filter out electromagnetic interference in a signal received by the wireless network card.

Preferably, the network communication module is a wired network card.

Compared with the prior art, the present invention has the following advantages: a network communication module receives an operation instruction and sends the operation instruction to a controller; and the controller controls a purification processing module to work according to the operation instruction; since the network communication module is used, a user may connect a purifier through a network, thereby implementing remote control and operation of the purifier and bringing convenience to the user.

### BRIEF DESCRIPTION OF THE DRAWINGS

FIG. 1 is a structural diagram of an ion air purifier in a closed state according to the present invention;

FIG. 2 is a structural diagram of the ion air purifier as shown in FIG. 1 in a median opened state;

FIG. 3 is a structural diagram of the ion air purifier as shown in FIG. 1 in a fully-opened state;

FIG. 4 is a block diagram of the principles of the first embodiment of a network air purifier provided by the present invention;

FIG. 5 is a block diagram of the principles of the second embodiment of a network air purifier provided by the present invention;

FIG. 6 is a block diagram of the principles of the third embodiment of a network air purifier provided by the present invention; and

FIG. 7 is a block diagram of the principles of the fourth embodiment of a network air purifier provided by the present invention.

### DETAILED DESCRIPTION

The core of the present invention is to provide an ion air purifier, which ensures an optimized distance between adsorption plates while facilitating cleaning.

The present invention will be explained in further details below with reference to the accompanying drawings and specific embodiments so as to enable those skilled in the art to better understand the technical solutions of the present invention.

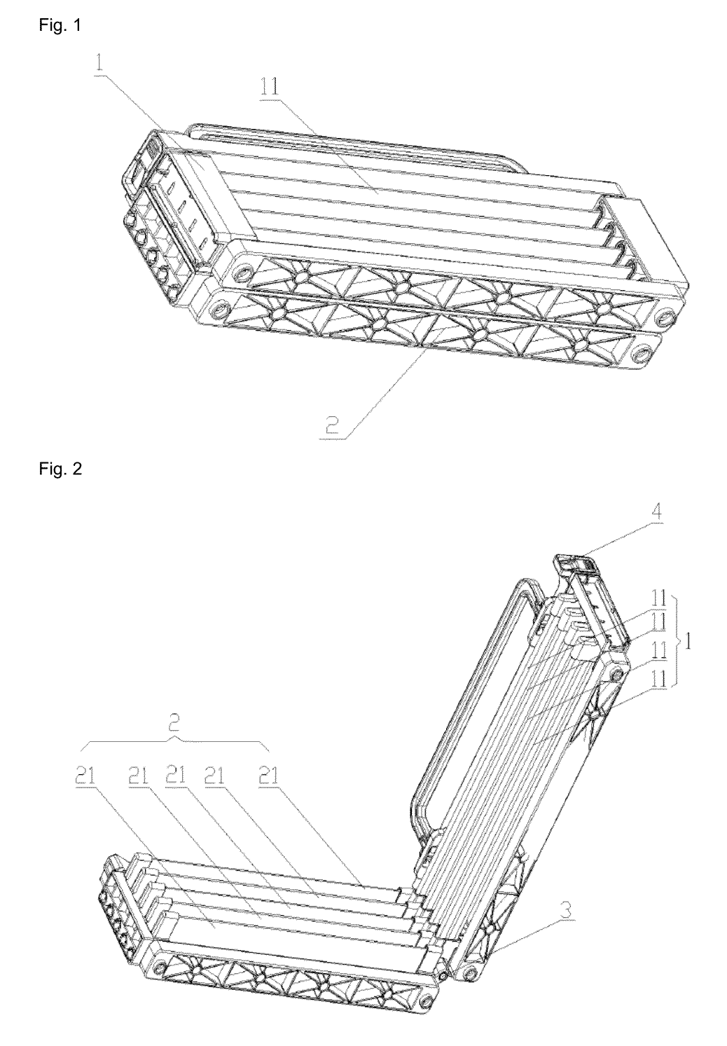

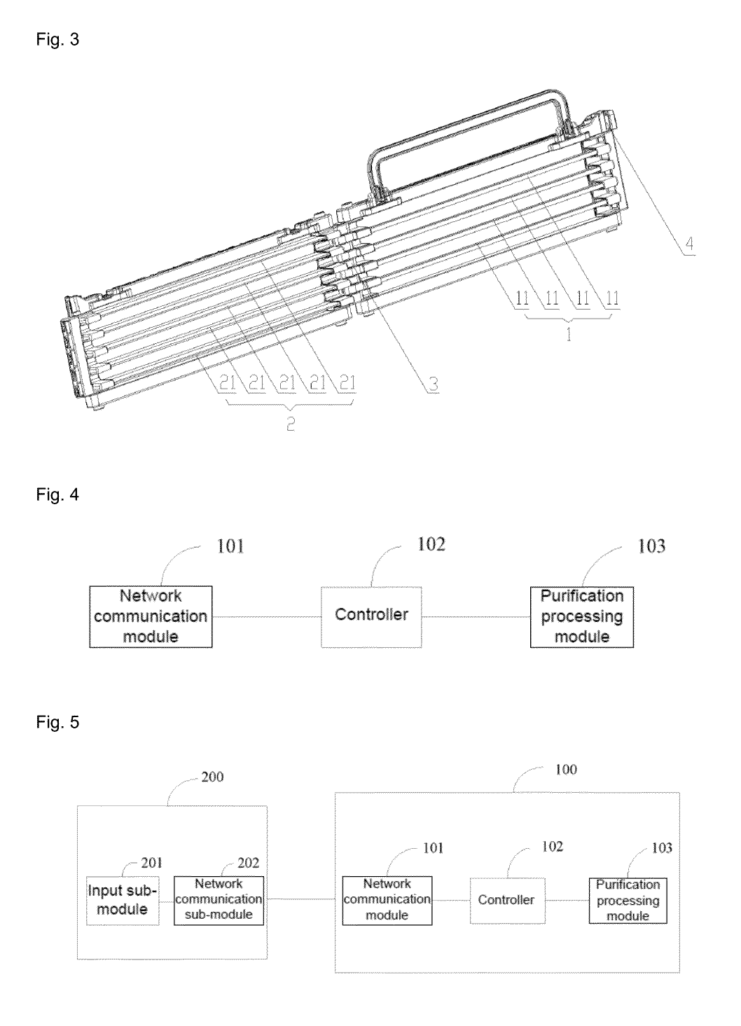

Please refer to FIG. 1 to FIG. 3, in which FIG. 1 is a structural diagram of an ion air purifier in a closed state according to the present invention, FIG. 2 is a structural diagram of the ion air purifier as shown in FIG. 1 in a median opened state; and FIG. 3 is a structural diagram of the ion air purifier as shown in FIG. 1 in a fully-opened state.

In a specific embodiment, the present invention provides an ion air purifier and the ion air purifier comprises a collector module 1 and a repeller module 2, which are connected in an openable and closable manner, wherein several collector plates 11 parallel to each other are uniformly distributed on the collector module 1, and several repeller plates 21 parallel to each other are uniformly distributed on the repeller module 2; the collector module and the repeller module are isolated electrically, and each collector plate 11 and each repeller plate 21 are alternately arranged when the two plates are in a closed state. When the collector module 11 and the repeller module 2 are in a closed state, the collector plates 11 are combined with the repeller plates 21, and the distance between adjacent collector plates and repeller plates is a set optimized distance. When the collector module 1 and the repeller module 2 are in an opened state, the collector plates 11 are separated from the repeller plates 21, and the distance between two adjacent collector plates 11 and the distance between two adjacent repeller plates 21 are both increased, thereby not only ensuring the optimized distance between the adjacent collector plates and repeller plates, but also facilitating cleaning of the modules.

The collector module further comprises a rotating shaft 3, and the rotating shaft 3 is provided on one end of the collector module 1. The repeller module 2 may be opened relative to the collector module 1 around the rotating shaft 3 under the action of an external force, thereby opening the two plates to facilitate cleaning, while preventing the two plates from being separated to facilitate locating when the plates are installed again.

In theory, the collector module is not limited to that provided with the rotating shaft 3 necessarily as long as the collector module 1 and the repeller module 2 can be connected detachably. For example, an appropriate location on the collector module 1 may be also slotted, and the repeller module 2 is installed in the collector module 1 in an insertion manner via the installation slot.

A fixing member 4 is provided on one end of the collector module 3 away from the rotating shaft 3 and the collector module 1 and the repeller module 2 are fixedly connected via the fixing member 4. In this way, the two plates are fixed by the fixing member 4 when the collector module 1 and the repeller module 2 are in the opened state, thus preventing the two plates from being opened irregularly and improving the reliability in installing the two plates.

The fixing member 4 may be also detachably installed on the collector module 1 and the repeller module 2.

The collector module is not limited to the structure with one end provided with the rotating shaft 3 and the other end provided with the fixing member 4, as long as the collector module 1 and the repeller module 2 are detachable. For example, fixing members 4 may be also provided on two ends of the collector module. The two plates are separated when the two fixing members 4 are opened, and the two plates are fixedly connected by the two fixing members 4 when the closed.

The fixing member 4 may be a hasp, and may be also other detachable connectors conventionally used in the field, such as a bolt connector etc.

An interval between the adjacent collector plates 11 is 3 mm to 20 mm, and an interval between the adjacent repeller plates 21 is 3 mm to 20 mm so that the distance between two adjacent absorption plates is an optimum value when the two plates are closed.

Preferably, an interval between the central lines of the adjacent collector plates 11 is 13 mm, and an interval between the central lines of the adjacent repeller plates 21 is 13 mm when the modules are fully unfolded.

Preferably, an interval between the central lines of a collector and a repeller is 6.5 mm when the modules are closed.

Generally, the plate thickness of each plate is preferably 1 mm.

It needs to be pointed out that an interval between the plates is the distance between the central lines of the two plates in the thickness direction.

Referring to FIG. 4, the figure is a block diagram of the principles of the first embodiment of a network air purifier provided by the present invention.

The present embodiment provides a network air purifier, comprising a network communication module 101, a controller 102 and a purification processing module 103.

The network communication module 101 is configured to receive an operation instruction via a network, and send the received operation instruction to the controller 102.

The controller 102 is configured to send a control signal to the purification processing module 103 according to the operation instruction sent by the network communication module 103, so as to control the purification processing module 103 to perform purification processing.

The purification processing module 103 is configured to perform air purification processing according to the control signal sent by the controller 102.

For example:

the network communication module 101 receives an operation instruction of “start device”, and sends the operation instruction to the controller 102.

The controller 102 sends a control signal of “start device” to the purification processing module 103 according to the received operation instruction, and the purification processing module 103 starts to work.

At the moment, the network communication module 101 further receives an operation instruction of “dehumidification and purification” and sends the operation instruction to the controller 102.

The controller 102 sends a control signal of “dehumidification and purification” to the purification processing module 103 according to the received operation instruction, and the purification processing module 103 starts to perform dehumidification.

The present embodiment has the following advantages: in the present embodiment, the network communication module receives an operation instruction, and sends the operation instruction to the controller and the controller controls the purification processing module to work according to the operation instruction. Since the network communication module is used, a user may connect the purifier through a network, thereby implementing remote control and operation of the purifier and bringing convenience to the user.

Referring to FIG. 5, the figure is a block diagram of the principles of the second embodiment of a network air purifier provided by the present invention.

In the network air purifier provided by the present embodiment, the network air purifier 100 further comprises a remote control terminal 200.

The remote control terminal 200 comprises an input sub-module 201 and a network communication sub-module 202.

The input sub-module 201 is configured to receive an operation instruction inputted by a user, and send the operation instruction to the network communication sub-module 202.

The network communication sub-module 202 is configured to connect the network air purifier 100 through a network, and send the operation instruction to the network communication module 101 of the purifier 100.

For example:

the network air purifier 100 further comprises a remote control terminal, such as a remote controller, and the remote control terminal may be also a Personal Computer

(PC), a Bluetooth device, or a mobile phone. Description will be provided below by taking a remote controller as an example.

The remote control terminal comprises an input sub-module 201, such as input devices comprising a button, a keyboard, and a touch screen etc.

The input sub-module 201 inputs an operation instruction for the network air purifier.

For example, the input sub-module selects a button of “start device”, or inputs an operation instruction of “start device” or clicks on an icon of “start device”.

The input device 201 sends the operation instruction of “start device” to a network communication sub-module 202 of the remote controller.

The network communication sub-module 202 is connected with a network communication module 101 of the network air purifier 100, and sends the operation instruction “start device” to the network communication module 101 of the network air purifier 100, thereby controlling the air purifier to be started to perform a purification operation.

The present embodiment has the following advantages: the remote control terminal is connected with the network air purifier, a user inputs an operation instruction through an input module of the remote control terminal, and the operation instruction is sent to the network communication module of the network air purifier via the network communication sub-module of the remote control terminal, thereby enabling a remote device to control and operate the air purifier remotely and bringing convenience to the user.

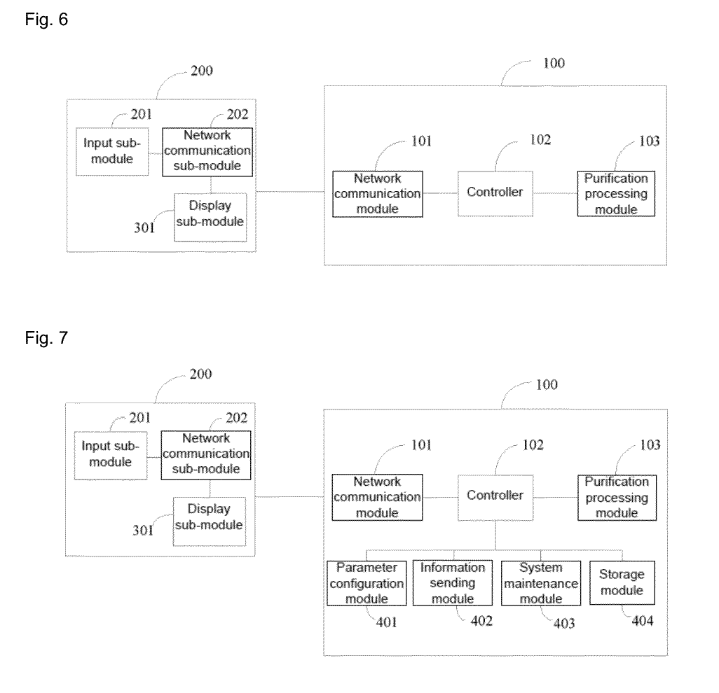

Referring to FIG. 6, the figure is a block diagram of the principles of the third embodiment of a network air purifier provided by the present invention.

In the network air purifier provided by the present embodiment, the remote control terminal 200 further comprises a display sub-module 301.

The display sub-module 301 is configured to display purification state information sent by the network communication module 101 of the network air purifier 100 to the network communication sub-module 202.

The purification state information is information for recording a device working state, which is generated when the purification processing module 103 of the network air purifier 100 performs the purification processing, or hardware alarm information of the network air purifier 100.

For example:

The purification processing module 103 of the network air purifier 100 sends state information “dehumidification and purification” to the display sub-module 301 on the remote control terminal 200 through the network communication module 101 during a working process. The display sub-module displays “dehumidification and purification” as a current state of the network air purifier 100.

Alternatively, alarm information indicating that “a purification component needs to be replaced” of the purification processing module 103 is sent to the display sub-module 301 to be displayed, thereby prompting a user to perform replacement.

The present embodiment has the following advantages: the display sub-module is added on the basis of the second embodiment of the network air purifier; the purification state information sent by the network communication module of the network air purifier to the network communication sub-module is displayed by the display sub-module, thereby monitoring a state of the network air purifier, providing a more friendly interface for a user, and facilitating the user to know a working state of the purifier.

Referring to FIG. 7, the figure is a block diagram of the principles of the fourth embodiment of a network air purifier provided by the present invention.

The network air purifier provided by the present embodiment further comprises a parameter configuration module 401; an information sending module 402, a system maintenance module 403 and a storage module 404.

The parameter configuration module 401 is configured to provide an interactive interface of the network air purifier 100 to set device parameters of the network air purifier. The interactive interface may be in mode of a webpage, and a user configures the network air purifier 100 via the webpage. The device parameters of the network air purifier are a network configuration parameter and a purification working parameter of the network air purifier 100. The purification working parameter is a parameter which is required when the purification processing module 103 of the network air purifier 100 operates.

The information sending module 402 is configured to send the purification state information of the purification processing module 103 to the remote control terminal 200 through the network communication module 101. The remote control terminal is provided with a display module 301 to display the received purification state information. The purification state information may be also sent to a maintenance server via the network communication module, and recorded by the maintenance server.

The system maintenance module 403 is configured to perform a maintenance operation to the network air purifier 100 according to system maintenance information received by the network communication module 101.

The system maintenance information comprises the purification working parameter of the network air purifier.

The storage module 404 is configured to store the purification state information sent by the purification processing module 103.

The present embodiment has the following advantages: by adding the parameter configuration module, a user may log in a webpage remotely, thereby implementing a graphical operation of the user; by adding the information sending module, a failure of the network air purifier may be monitored remotely, so that a potential failure may be discovered in time; by adding the system maintenance module, a software parameter etc. of the network air purifier may be upgraded and maintained remotely, and by adding the storage module, purification state information of the purification processing module may be saved locally so that a failure reason may be judged according to the state information when the device fails.

The above embodiments are only preferred embodiments of the present invention, but are not intended to limit the present invention in any form. Although the present invention has been disclosed as above by the preferred embodiment, the preferred embodiments are not used for limiting the present invention. Any person skilled in the art may make many possible changes and modifications to the technical solutions of the present invention, or change the technical solutions of the present invention into equivalent embodiments having equivalent changes by taking advantage of the method and the technical content disclosed above without departing from the scope of the technical solutions of the present invention. Therefore, any simple alternations, equivalent changes and modifications made to the above embodiments according to the technical essence of the present invention without departing from the content of the technical solutions of the present invention will also belong to the scope of protection of the technical solutions of the present invention.

1. An ion air purifier, comprising a collector module (1) and a repeller module (2), which are connected in an openable and closable manner; several collector plates (11) parallel to each other are uniformly distributed on the collector module (1), and several repeller plates (21) parallel to each other are uniformly distributed on the repeller module (2); the collector module and the repeller module are isolated electrically, and each collector plate (11) and each repeller plate (21) are alternately arranged when the two plates are in a closed state.

2. The ion air purifier according to claim 1, wherein a rotating shaft (3) is provided on one end of the collector module (1), and the repeller module (2) is opened relative to the collector module (1) around the rotating shaft (3).

3. The ion air purifier according to claim 2, wherein one end of the collector module away from the rotating shaft (3) is provided with a fixing member (4), and the collector module (1) and the repeller module (2) are fixedly connected via the fixing member (4).

4. The ion air purifier according to claim 3, wherein the fixing member (4) is a hasp.

5. The ion air purifier according to claim 3, wherein the fixing member (4) is detachably installed on the collector module (1) and the repeller module (2).

6. The ion air purifier according to claim 1, wherein when the collector module (1) and the repeller module (2) are in a closed state, adjacent collector plates (11) and repeller plates (21) are mutually insulated with a potential difference, and the ratio of the potential difference to the distance between the adjacent collector plates (11) and repeller plates (21) is smaller than 10⁷ V/m.

7. A network air purifier, comprising a network communication module, a controller and a purification processing module;
the network communication module is configured to receive an operation instruction via a network, and send the received operation instruction to the controller;
the controller is configured to send a control signal to the purification processing module according to the operation instruction sent by the network communication module and control the purification processing module to perform a purification operation; and
the purification processing module is configured to perform air purification processing according to the control signal sent by the controller.

8. The purifier according to claim 7, wherein the purifier further comprises a remote control terminal;
the remote control terminal comprises an input sub-module and a network communication sub-module;
the input sub-module is configured to receive an operation instruction inputted by a user, and send the operation instruction to the network communication sub-module; and
the network communication sub-module is configured to connect the purifier through a network, and send the operation instruction to the network communication module of the purifier.

9. The purifier according to claim 8, wherein the remote control terminal further comprises a display sub-module;
the display sub-module is configured to display purification state information sent by the network communication module of the purifier to the network communication sub-module; and
the purification state information is information for recording a device working state, which is generated when the purification processing module of the purifier performs the purification processing, or hardware alarm information of the purifier.

10. The purifier according to claim 7, wherein the purifier further comprises a parameter configuration module;
the parameter configuration module is configured to provide an interactive interface of the purifier to set device parameters of the purifier;
the device parameters of the purifier are a network configuration parameter and a purification working parameter of the purifier; and
the purification working parameter is a parameter which is required when the purification processing module of the purifier operates.

11. The purifier according to claim 7, wherein the purifier further comprises an information sending module;
the information sending module is configured to send the purification state information to the remote control terminal through the network communication module; the remote control terminal is provided with a display module to display the received purification state information; and
the purification state information is sent to a maintenance server via the network communication module and recorded by the maintenance server.

12. The purifier according to claim 7, wherein the purifier further comprises a system maintenance module;
the system maintenance module is configured to perform a maintenance operation to the purifier according to system maintenance information received by the network communication module; and
the system maintenance information comprises the purification working parameter of the purifier.

13. The purifier according to claim 7, wherein the purifier further comprises a storage module; and
the storage module is configured to store the purification state information sent by the purification processing module.

14. The purifier according to claim 7, wherein the network communication module is a wireless network card.

15. The purifier according to claim 14, wherein the purifier further comprises a filter module; and
the filter module is configured to filter out electromagnetic interference in a signal received by the wireless network card.

16. The purifier according to claim 7, wherein the network communication module is a wired network card.

17. The ion air purifier according to claim 2, wherein when the collector module (1) and the repeller module (2) are in a closed state, adjacent collector plates (11) and repeller plates (21) are mutually insulated with a potential difference, and the ratio of the potential difference to the distance between the adjacent collector plates (11) and repeller plates (21) is smaller than 10⁷ V/m.

18. The ion air purifier according to claim 3, wherein when the collector module (1) and the repeller module (2) are in a closed state, adjacent collector plates (11) and repeller plates (21) are mutually insulated with a potential difference, and the ratio of the potential difference to the distance between the adjacent collector plates (11) and repeller plates (21) is smaller than 10⁷ V/m.

19. The ion air purifier according to claim 4, wherein when the collector module (1) and the repeller module (2) are in a closed state, adjacent collector plates (11) and repeller plates (21) are mutually insulated with a potential difference, and the ratio of the potential difference to the distance between the adjacent collector plates (11) and repeller plates (21) is smaller than 10⁷ V/m.

20. The ion air purifier according to claim 5, wherein when the collector module (1) and the repeller module (2) are in a closed state, adjacent collector plates (11) and repeller plates (21) are mutually insulated with a potential difference, and the ratio of the potential difference to the distance between the adjacent collector plates (11) and repeller plates (21) is smaller than 10⁷ V/m.
